SEQUENCE LISTING (2 sequences)

SEQ ID NO: 1 - DNA, 601 bp, Homo sapiens
agctgcaatgcaggaagactccctgatgtgatcatgtgtctcaccctttcaagctgaaagcaacagtacagacgatgagaccgacgatcccaggacgtatcagaaatatggttacattggaacacatgagtaccctcatttctctcatagacccagcacactccaggcagcatccaccccacaggcagaagaggactggacctgcaccatcctctgaatgccagtgcatcttgggggaaagggctccagtgttatctggaccagttccttcattttcaggtgggactcttgatccagagargacaaagctcctcagtgagctggtgtataatccaggacagaacccaggtctcctgactcctggccttctatgccctctatcctatcatagataacattctccacagcctcacttcattccacctattctctgaaaatattccctgagagagaacagagagatttagataagagaatgaaattccagccttgactttcttctgtgcacctgatgggagggtaatgtctaatgtattatcaataacaataaaaataaagcaaataccatttattgggtgtttattaacttcaaggcacagag

SEQ ID NO: 2 - DNA, 601 bp, Homo sapiens
catccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttyggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagagaatgaaattgctgaaagatctttcaggactctacctcatatcagtttgctagcagaaatctagaagactgtc

(19) United States
(12) Patent Application Publication
(10) Pub. No.: US 2011/0008290 A1
(43) Pub. Date: Jan. 13, 2011

(54) METHOD FOR PREDICTING THE THERAPEUTIC RESPONSIVENESS OF PATIENTS TO A MEDICAL TREATMENT WITH AN INTERFERON

(76) Inventor: David Brassat, Toulouse Cedex (FR)

Correspondence Address: YOUNG & THOMPSON, 209 Madison Street, Suite 500, Alexandria, VA, 22314, US

(21) Appl. No.: 12/741,885

(22) PCT Filed: Nov. 7, 2008
(86) PCT No.: PCT/EP2008/065130

§ 371 Date: Sep. 13, 2010

(30) Foreign Application Priority Data
Nov. 9, 2007 (EP) 07301538.0

(51) Int. Cl. A61K 38/21; G01N 33/53; A61P 25/28

(52) U.S. Cl. 424/85.6; 436/94

(57) ABSTRACT

A method for predicting a response of a patient to a medical treatment with interferon, wherein the method includes detecting the allele identity of a polymorphism in the OAS1 gene in a nucleic acid sample previously obtained from the patient, and optionally detecting the allele identity

### FIELD OF THE INVENTION

The present invention relates to a method for predicting the response to a medical treatment of a disease with an Interferon (IFN) agent, including a medical treatment of a disease selected from Multiple Sclerosis, hepatitis and cancer

### BACKGROUND OF THE INVENTION

Interferons in a broad meaning are extracellular messengers mediating reactivity of hosts and evolutionally conserved protein families that are released in a relatively small size from cells. Interferons are released from interferon-producing cells in response to stimulation by viruses, double-stranded RNAs, various microorganisms, or cytokines such as TNF or IL1, and then bind to surfaces of neighboring cells with interferon receptors. Thereafter, interferons induce synthesis of various proteins so that reactivity and homeostasis of hosts are maintained by consecutive signaling in the cells. Therefore, interferons act as antiviral, antiproliferative, and immune signaling proteins in the bodies and have direct antiproliferation effects on cancer cells, and thus, have received much attention as therapeutic agents [Postka S., Langer J. A. and Zoon K. C. (1987) Interferons and their actions, Annu. Rev. Biochem. 56:727-777].

Interferons are small inducible proteins secreted by nucleated cells in response to viral infection or other stimuli, and act in a paracrine fashion on other cells in their immediate vicinity. The type 1 interferons, which include about 15 cytokines (thirteen isotypes of IFNα one IFNβ and one IFNω), all share a common receptor present on most cell types and are made up of two subunits (IFNAR-1 and IFNAR-2). Upon binding, interferons induce complex intracellular processes resulting in the transcriptional activation and production of IFN-sensitive genes (ISG), which display multiple antiviral, antiproliferative, and immunomodulatory activities⁴. In addition to MS, therapeutic applications of type I interferons include hepatitis and cancer. Among them, interferon betas belonging to the type 1 interferon are proteins that exhibit species specificity. Interferon betas are also called as fibroblast interferons considering their sources and as pH2-stable interferons considering biological characteristics. Interferon betas bind to the same receptors of cell surfaces, together with interferon alphas belonging to the type 1 interferon, and then induce transcription of antiviral factors in response to a consecutive cell signaling system. Currently, interferon betas are manufactured by gene recombination technology. Recombinant interferon betas are known to be effective in delaying the progression of multiple sclerosis in patients with the signs of the disease and relieving the pains of the disease. Furthermore, recombinant interferon betas are widely used as therapeutic agents for multiple sclerosis, and at the same time are effective in nonspecific regulation of human immune response, immune response to viral infection, and anti-proliferation of cancer cells.

The most significant development in Multiple Sclerosis (MS) therapeutics has been the approval over 10 years ago of recombinant interferon beta (IFNβ) for the relapsing-remitting form of the disease3. Multiple sclerosis is a severe disorder of the central nervous system (CNS) characterized by chronic inflammation, myelin loss, gliosis, varying degrees of axonal and oligodendrocyte pathology, and progressive neurological dysfunction. MS clusters with the so-called complex genetic diseases, a group of common disorders characterized by modest disease risk heritability and multifaceted gene-environment interactions. MS is the most common cause of acquired neurological disability arising between early to middle adulthood, affecting approximately 2 million people worldwide. No curative therapy is currently available, and approximately 80-90% of afflicted individuals are ultimately disabled.

In MS, IFNβ has been shown to decrease up to 30% of clinical relapses, reduce brain MRI activity and lesion load, and possibly slow progression of disability

However, with an increasing number of interferon-treated patients with time, it has been shown that a substantial proportion of patients did not respond to interferon treatments, including to beta interferon treatments. This is particularly true for patients which are affected with multiple sclerosis and are treated with beta interferon.

Further, medical treatment of patients with interferons often cause various side effects that are sometimes deleterious to the patients health.

Illustratively, despite a measurable influence on patients' quality of life, the effect of treatment with beta interferon, including in patients affected with MS, is partial, and a substantial proportion of patients are not responders.

Furthermore, IFNβ therapy has been associated with a number of adverse reactions, and its long-term impact on disease progression remains unknown. Notably, medical treatments with beta interferon very often cause injection site reactions and, in a more limited number of cases, may cause injection site necrosis, inflammation, pain, hypersensitivity, as well as various non-specific reactions. It has also been reported side-effects including flu-like symptom complex, along with fever, chills, myalgia, malaise and sweating. It has also been reported depression, anxiety, emotional lability, depersonalization and even suicide attempts, Irrespective of their frequency, inflammation, pain and necrosis are medically considered as severe side effects associated with beta interferon treatments.

Hence, in the absence of clinical, neuro-radiological and/or immunological predictive markers of response to interferons, including beta interferon, and given that a significant portion of patients have often relatively benign forms of the disease to be treated, the question remains for medical practitioners to whom and when to recommend interferon treatment, when side effects, inconvenience, and cost of the drug are significant. There is thus a need in the art for reliable markers allowing the discrimination between (i) disease-affected patients to whom an interferon treatment will bring a medical benefit, which patients may also be termed “responders” and (ii) disease-affected patients to whom an interferon treatment will bring no medical benefit, but who may undergo interferon-induced side effects, which patients may also be termed “non-responders”.

### SUMMARY OF THE INVENTION

An object of the invention consists of a method, in particular of an in vitro method, for predicting the response of a patient to a treatment with a IFN agent, the said method comprising a step of determining the presence or absence of a guanine at a biallelic marker located at position 301 of the OAS1 gene sequence portion of SEQ ID N^{o}1 from said patient, wherein the presence of a guanine at the said biallelic marker location in both copies of said OAS1 gene of said patient is indicative of a an increased risk that the said patient consists of a good responder to interferon treatment with respect to standard responsiveness.

In certain embodiments of the method, the said method further comprises a step of determining the presence or absence of a tyrosine at a biallelic marker located at position 301 of the TRAIL gene sequence portion of SEQ ID N^{o}2 from said patient, wherein the presence of a tyrosine at the said biallelic marker location in both copies of said TRAIL gene of said patient is indicative of a an increased risk that the said patient consists of a good responder to interferon treatment with respect to standard responsiveness.

In preferred embodiment the patient suffers from Multiple Sclerosis disorder, hepathitis or cancer, and in more preferably embodiment the patient suffers from Multiple Sclerosis disorder.

In certain embodiments the interferon treatment consists of a treatment with recombinant human interferon type I (IFNβ or INFα).

Most preferably, the interferon treatment consists of a treatment with a recombinant human interferon-beta (IFNβ) when patient suffer from Multiple Sclerosis disorder.

The present invention also relates to the use of an interferon agent for the manufacture of a medicament intended for treating a patient, wherein said patient possesses a G/G homozygosity at the biallelic marker located at position 301 in the nucleic acid sequence of SEQ ID N^{o}1 from the said patient.

This invention also pertains to the use of an interferon agent for the manufacture of a medicament intended for treating a patient, wherein said patient possesses:
(i) a G/G homozygosity at the biallelic marker located at position 301 in the nucleic acid sequence of SEQ ID N^{o}1 from the said patient, and
(i) a TIT homozygosity at the biallelic marker located at position 301 in the nucleic acid sequence of SEQ ID N^{o}2 from the said patient.

In preferred embodiments, the said patient is affected with Multiple Sclerosis disorder, hepatitis or cancer, and in more preferably embodiment the patient suffers from Multiple Sclerosis disorder.

In a preferred embodiments, the interferon treatment consists of a treatment with recombinant human interferon type I (IFNβ or INFα).

Most preferably, the interferon treatment consists of a treatment with a recombinant human interferon-beta (IFNβ) when patient suffer from Multiple Sclerosis disorder.

The present invention also relates to a method for treating a patient likely to respond to treatment with an interferon agent, which method comprises the steps of:
a) determining whether a patient is a responder or a non responder to a treatment with interferon agent, by performing the above mentioned in vitro method by determining the presence or absence of a a guanine at position −301 of the OAS1 gene of said patient, and
b) administering a therapeutically effective amount of an interferon agent to the said patient, if the said patient has been determined as consisting of a good responder, at step a) above, i.e. if said patient does simultaneously carry a guanine at position 301 of the OAS1 gene portion of SEQ ID N^{o}1 in both copies of said OAS1 gene of said patient

In certain embodiments, the said patient is affected with Multiple Sclerosis disorder, hepathitis or cancer. In most preferred embodiments, the said patient is affected with Multiple Sclerosis disorder.

In preferred embodiments, the interferon treatment consists of a treatment with a recombinant human interferon type I (IFNβ or INFα) treatment. Most preferably, the interferon treatment consists of a treatment with a recombinant human interferon-beta (IFNβ) when the patient suffers from Multiple Sclerosis disorder.

### DETAILED DESCRIPTION OF THE INVENTION

The present inventors have assayed for a possible statistical linkage between (i) specific polymorphisms contained in various candidate genes from patients in need of a medical treatment with an interferon agent and (ii) the ability of the said patients to react positively to the said medical treatment.

By genotyping nucleic acid samples originating from (i) patients consisting of good responders to a medical treatment with an interferon agent, especially with β interferon, and (ii) patients consisting of non-responders to a treatment with the said interferon agent, the inventors have found that there is a high statistical linkage between (1) a specific allele of specific SNP biallelic markers contained in the sequence of the OAS1 gene or of the TRAIL gene, respectively, and (2) the ability of the corresponding patients to respond positively to the said medical treatment.

As disclosed in the examples herein, the inventors have screened nucleic acid samples originating from a well characterized cohort of patients affected with Multiple Sclerosis (MS) which were treated with β-IFN (i.e. “a well characterized IFNβ-treated MS dataset”) to assess the pharmacogenomic effects of single nucleotide polymorphisms (SNP) in eight ISG genes (for “Interferon-Sensitive Gene”) previously reported to be implicated in the modulation of IFNα or β treatments. Significant associations between IFN β treatment response and OAS1 and TRAIL polymorphisms have been determined.

More precisely, the inventors have genotyped eight ISG genes and thirteen single nucleotide polymorphisms (SNPs) contained in these ISG genes, in a large cohort of Caucasian patients with Multiple Sclerosis and treated with Betaferon® (Interferon β-1b by Schering), with Avonex® (Interferon β-1a by Biogen Idec) or with Rebif® ((Interféron β-1a by Serono). For each ISG gene, SNP biallelic markers, as well as haplotypes thereof, were tested for associations between the existence of specific alleles and the kind of clinical outcome (responder or non-responder to treatment). Patients were followed-up prospectively at least for 2 years since initiation of therapy. Clinical data such as the EDSS (for “Expanded Disability Status Scale”) was recorded every 3 months. The individual alleles were compared and tabulated under Genotype 1 (binary variable where 11 and 12 are compared to 22; so allele 1+ vs. allele 1−) and Genotype 2 (binary variable where 12 and 22 are compared to 11; so allele 2+ vs. allele 2−). One model was used for each gene polymorphism. Sex, disease type, disease duration at treatment onset, interferon type, and number of relapse before treatment onset were included in each model as they were potential cofounders. In a secondary analysis, the inventors looked at a potential interaction between genes associated with an increased risk for non-response (IRNR) to IFNβ treatment.

The inventors have now identified a specific SNP biallelic marker contained in the genomic sequence of the OAS1 gene wherein an homozygosity for one of the two alleles is linked to a phenotype of good response to a medical treatment with an interferon agent.

The inventors have thus identified that the SNP consisting of 301A/G (NCBI ref. rs2660) in the OAS1 gene may be useful to predict the response to a treatment with a IFN agent.

More precisely, it has been found according to the invention that a good response of a patient to a medical treatment with an interferon agent may be predicted when a G/G homozygosity at the SNP biallelic marker located at position 301 of the nucleic acid sequence of SEQ ID N^{o}1 that is found in a nucleic acid sample from the said patient, which sequence SEQ ID N^{o}1 consists of a sequence portion of the OAS1 genomic sequence. In the nucleic acid sequence of SEQ ID N^{o}1, the SNP biallelic marker is bordered by a 5′-end sequence and a 3′-end sequence having each 300 nucleotides in length, respectively. The nucleic acid sequence of SEQ ID N^{o}1 which defines the SNP biallelic marker of interest is known per se and is notably referred to n^{o} rs2660 in the “Single Nucleotide Polymorphism” database of the NCBI.

The inventors have also identified a specific SNP biallelic marker contained in the genomic sequence of the TRAIL gene wherein an homozygosity for one of the two alleles is linked to a phentotype of good response to a medical treatment with an interferon agent.

The inventors have thus also identified that the SNP consisting of 301C/T (NCBI ref. rs2660) in the TRAIL gene may be useful to predict the response to treatment with a IFN agent.

More precisely, it has been found according to the invention that a good response of a patient to a medical treatment with an interferon agent may be predicted when a T/T homozygosity at the SNP biallelic marker located at position 301 of the nucleic acid sequence of SEQ ID N^{o}2 that is found in a nucleic acid sample from the said patient, which sequence SEQ ID N^{o}2 consists of a sequence portion of the TRAIL genomic sequence. In the nucleic acid sequence of SEQ ID N^{o}2, the SNP biallelic marker is bordered by a 5′-end sequence and a 3′-end sequence having each 300 nucleotides in length, respectively. The nucleic acid sequence of SEQ ID N^{o}2 which defines the SNP biallelic marker of interest is known per se and is notably referred to n^{o} rs1131532 in the “Single Nucleotide Polymorphism” database of the NCBI.

The potential relevance of TRAIL, which encodes a cytokine that belongs to the tumour necrosis factor (TNF) ligand family, as a biological marker for predicting response to IFNβ was previously suggested by a study reporting that patients with high concentration of soluble TRAIL in the serum have a better response to—therapy (Wandinger et al., 2003). In addition, a gene expression study of peripheral blood mononuclear cell (PBMC) demonstrated that responders experienced an early and sustained induction of TRAIL (Wandinger et al., 2003). These inventors had already found that patients C/C homozygote for the SNP rs1131532, had an increased risk to be non responders as compare to C/T and T/T patients (OR=2.1, p=0.041). But there is a need for an alternative solution in order to find a new bio- and genetic markers to predict the response to treatment with a INF-beta agent.

The inventors have demonstrated that the SNP consisting of 301A/G (rs2660) in the OAS1 gene was significantly associated with the response of the patient to a treatment with an interferon agent, and more specifically to a treatment with a IFNβ agent.

### Definitions:

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term “gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. In particular, the term gene may be intended for the genomic sequence encoding a protein, i.e. a sequence comprising regulator, promoter, intron and exon sequences.

As used herein, the term “oligonucleotide” refers to a nucleic acid, generally of at least 10, preferably at least 15, and more preferably at least 20 nucleotides, preferably no more than 100 nucleotides, still preferably no more than 70 nucleotides, and which is hybridizable to a genomic DNA, cDNA, or mRNA. Oligonucleotides can be labelled according to any technique known in the art, such as with radiolabels, fluorescent labels, enzymatic labels, sequence tags, etc. A labelled oligonucleotide may be used as a probe to detect the presence of allelic variants of TNF nucleic acid. Alternatively, oligonucleotides (one or both of which may be labelled) can be used for amplifying a region of a TNF nucleic acid, for instance by PCR (Saiki et al., 1988), to detect the presence of an allelic variant. Generally, oligonucleotides are prepared synthetically, preferably on a nucleic acid synthesizer. Accordingly, oligonucleotides can be prepared with non-naturally occurring phosphoester analog bonds, such as thioester bonds, etc.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., 1989).

The conditions of temperature and ionic strength determine the “stringency” of the hybridization. For preliminary screening for homologous nucleic acids, low stringency hybridization conditions, corresponding to a Tm (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tm, e.g., 40% formamide, with 5× or 6×SCC. High stringency hybridization conditions correspond to the highest Tm, e.g., 50% formamide, 5× or 6×SCC. SCC is a 0.15 M NaCl, 0.015 M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA: RNA, DNA: RNA, DNA: DNA. For hybrids of greater than 100 nucleotides in length, equations for calculating Tm have been derived (see Sambrook et al., 1989, 9.50-9.51). For hybridization with shorter nucleic acids, i.e., oligonucleotides, the position of mismatches becomes more important, and the length of the oligonucleotide determines its specificity (see Sambrook et al., 1989 II.7-11.8). A minimum length for a hybridizable nucleic acid is at least about 10 nucleotides, preferably at least about 15 nucleotides, and more preferably the length is at least about 20 nucleotides.

In a specific embodiment, the term “standard hybridization conditions” refers to a Tm of 55° C., and utilizes conditions as set forth above. In a preferred embodiment, the Tm is 60° C. In a more preferred embodiment, the Tm is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

As used herein, an amplification primer is an oligonucleotide for amplification of a target sequence by extension of the oligonucleotide after hybridization to the target sequence or by ligation of multiple oligonucleotides which are adjacent when hybridized to the target sequence. At least a portion of the amplification primer hybridizes to the target. This portion is referred to as the target binding sequence and it determines the target-specificity of the primer. In addition to the target binding sequence, certain amplification methods require specialized non-target binding sequences in the amplification primer. These specialized sequences are necessary for the amplification reaction to proceed and typically serve to append the specialized sequence to the target. For example, the amplification primers used in Strand Displacement Amplification (SDA) include a restriction endonuclease recognition site 5′ to the target binding sequence (U.S. Pat. No. 5,455,166 and U.S. Pat. No. 5,270,184). Nucleic Acid Based Amplification (NASBA), self-sustaining sequence replication (3SR) and transcription based amplification primers require an RNA polymerase promoter linked to the target binding sequence of the primer. Linking such specialized sequences to a target binding sequence for use in a selected amplification reaction is routine in the art. In contrast, amplification methods such as PCR which do not require specialized sequences at the ends of the target, generally employ amplification primers consisting of only target binding sequence.

As used herein, the terms “primer” and “probe” refer to the function of the oligonucleotide. A primer is typically extended by polymerase or ligation following hybridization to the target but a probe typically is not. A hybridized oligonucleotide may function as a probe if it is used to capture or detect a target sequence, and the same oligonucleotide may function as a primer when it is employed as a target binding sequence in an amplification primer. It will therefore be appreciated that any of the target binding sequences disclosed herein for amplification, detection or quantisation of OAS1 gene may be used either as hybridization probes or as target binding sequences in primers for detection or amplification, optionally linked to a specialized sequence required by the selected amplification reaction or to facilitate detection.

As used herein, the term “OAS1 gene” denotes the human gene to which the methods of the invention can apply. The OAS1 gene maps to 12q24.1 and encodes for a key enzyme in the IFN□ and β regulation cascade, the 2′5′ oligoadenylate synthetase. After their binding to the IFNAR receptor and triggering of the JAK/STAT cascade, there is an activation of transcriptional factors that will interact with a promoter sequence called interferon stimulated response element (ISRE)4. The sequence of the OAS1 gene is notably available in the Genbank database under accession number AAC52140 (MIM:164350).

As used herein, the term “TRAIL gene” denotes the human gene to which the methods of the invention can also apply in combination with the OAS gene. TRAIL maps to 3q26 and encodes a cytokine that belongs to the tumour necrosis factor (TNF) ligand family. This protein preferentially induces apoptosis in transformed and tumour cells. TRAIL has been implicated in autoimmune diseases such as MS. It appears that TRAIL may be a paradigm of neuroinflammation pathogenesis complexity. In one hand, it has been postulated that TRAIL was a major player leading to central neural damage in the mouse model of MS. In the other hand, TRAIL may have an immunomodulatory function since mice lacking TRAIL experienced an increased susceptibility to autoimmunity. The mechanism could be an inhibition of autoreactive T cells activation, probably involving thymocyte apoptosis. Nevertheless, in humans, TRAIL does not appear to induce apoptosis of antigen specific T cells, rather TRAIL may inhibit T cell activation. This could be linked to a blockade of calcium influx. The sequence of the TRAIL gene is notably available in the Genbank database under the accession number P50591

The terms “mutant” and “mutation” mean any detectable change in genetic material, e.g. DNA, RNA, cDNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence. Generally a mutation is identified in a subject by comparing the sequence of a nucleic acid or polypeptide expressed by said subject with the corresponding nucleic acid or polypeptide expressed in a control population. A mutation in the genetic material may also be “silent”, i.e. the mutation does not result in an alteration of the amino acid sequence of the expression product.

The term “allele” is used herein to refer to variants of a nucleotide sequence. A biallelic polymorphism has two forms. Typically the first identified allele is designated as the original allele whereas other alleles are designated as alternative alleles. Diploid organisms may be homozygous or heterozygous for an allelic form.

The term “genotype” as used herein refers the identity of the alleles present in an individual or a sample. In the context of the present invention a genotype preferably refers to the description of the biallelic marker alleles present in an individual or a sample. The term “genotyping” a sample or an individual for a biallelic marker consists of determining the specific allele or the specific nucleotide carried by an individual at a biallelic marker.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A single nucleotide polymorphism is a single base pair change. Typically a single nucleotide polymorphism is the replacement of one nucleotide by another nucleotide at the polymorphic site. Deletion of a single nucleotide or insertion of a single nucleotide, also give rise to single nucleotide polymorphisms. In the context of the present invention “single nucleotide polymorphism” preferably refers to a single nucleotide substitution. Typically, between different genomes or between different individuals, the polymorphic site may be occupied by two different nucleotides.

The terms “biallelic polymorphism” and “biallelic marker” are used interchangeably herein to refer to a polymorphism having two alleles at a fairly high frequency in the population, preferably a single nucleotide polymorphism. A “biallelic marker allele” refers to the nucleotide variants present at a biallelic marker site.

As used herein, a SNP biallelic marker is defined as the variable nucleotide that is located at the center of the nucleic acid sequence in which this variable nucleotide is contained.

The terms “complementary” or “complement thereof” are used herein to refer to the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

The term “patient” refers to any subject (preferably human) afflicted with a disease likely to benefit from a treatment with an IFN agent, in particular a IFN-related disease.

The term “Interferon agent” or “IFN agent” encompasses type 1 interferon molecules selected form the group consisting of IFNα, IFNβ and IFNω. In certain preferred embodiments, an interferon agent is selected from the group consisting of IFNα and IFNβ. In other preferred embodiments, an interferon agent consists of an IFNβ, more preferably an IFNβ-1, and most preferably an IFNβ-1 selected from the group consisting of IFNβ-1a and IFNβ-1b.

A “responder” or “responsive” patient, or group of patients, to a treatment with a IFN agent, refers to a patient which is affected with a disease, or group of patients which are affected with a disease, who show(s) or will show a clinically significant relief in the disease when treated with a IFN agent. The disease clinical data may be assessed according to the standards recognized in the art, such as the “Disease Activity Score” (EDSS). The Kurtzke Expanded Disability Status Scale (EDSS) (Kurtzke, Neurology, 1983, 33:1444-52) is a method of quantifying disability in multiple sclerosis. The EDSS replaced the previous Disability Status Scales which used to bunch people with MS in the lower brackets.

The EDSS quantifies disability in eight Functional Systems (FS) and allows neurologists to assign a Functional System Score (FSS) in each of these. The Functional Systems are: pyramidal, cerebellar, brainstem, sensory, bowel and bladder, visual, cerebral, and other.

EDSS steps 1.0 to 4.5 refer to people with Multiple Sclerosis (MS) who are fully ambulatory. EDSS steps 5.0 to 9.5 are defined by the impairment to ambulation.

Clinical assessment of Multiple Sclerosis by EDSS according to Kurtzke is detailed in Table 1 hereunder.

TABLE 1
Kurtzke Expanded Disability Status Scale

| | |
|---|---|
| 0.0 | Normal neurological examination |
| 1.0 | No disability, minimal signs in one FS |
| 1.5 | No disability, minimal signs in more than one FS |
| 2.0 | Minimal disability in one FS |
| 2.5 | Mild disability in one FS or minimal disability in two FS |
| 3.0 | Moderate disability in one FS, or mild disability in three or four FS. Fully ambulatory |
| 3.5 | Fully ambulatory but with moderate disability in one FS and more than minimal disability in |
| | several others |
| 4.0 | Fully ambulatory without aid, self-sufficient, up and about some 12 hours a day despite |
| | relatively severe disability; able to walk without aid or rest some 500 meters |
| 4.5 | Fully ambulatory without aid, up and about much of the day, able to work a full day, may |
| | otherwise have some limitation of full activity or require minimal assistance; characterized by |
| | relatively severe disability; able to walk without aid or rest some 300 meters. |
| 5.0 | Ambulatory without aid or rest for about 200 meters; disability severe enough to impair full |
| | daily activities (work a full day without special provisions) |
| 5.5 | Ambulatory without aid or rest for about 100 meters; disability severe enough to preclude full |
| | daily activities |
| 6.0 | Intermittent or unilateral constant assistance (cane, crutch, brace) required to walk about |
| | 100 meters with or without resting |
| 6.5 | Constant bilateral assistance (canes, crutches, braces) required to walk about 20 meters |
| | without resting |
| 7.0 | Unable to walk beyond approximately five meters even with aid, essentially restricted to |
| | wheelchair; wheels self in standard wheelchair and transfers alone; up and about in wheelchair |
| | some 12 hours a day |
| 7.5 | Unable to take more than a few steps; restricted to wheelchair; may need aid in transfer; |
| | wheels self but cannot carry on in standard wheelchair a full day; May require motorized |
| | wheelchair |
| 8.0 | Essentially restricted to bed or chair or perambulated in wheelchair, but may be out of bed |
| | itself much of the day; retains many self-care functions; generally has effective use of arms |
| 8.5 | Essentially restricted to bed much of day; has some effective use of arms retains some self |
| | care functions |
| 9.0 | Confined to bed; can still communicate and eat. |
| 9.5 | Totally helpless bed patient; unable to communicate effectively or eat/swallow |
| 10.0 | Death due to MS. |

As intended herein “an increase risk to consist of a good (or non) responder to interferon treatment with respect to standard responsiveness” means that the probability that a patient, e.g. a patient affected with with MS, which is homozygous for the 301G/A haplotype will be responsive to treatment a IFN agent is higher, or alternatively lower, than that observed for a general population of patients with the same pathology, e.g. MS. As intended herein a “general population of patients” denotes a population of unselected patients, in particular as regards their OAS genotype. Preferably, the general population comprises enough patients so that the ratio of patients who respond to the treatment can be considered as statistically significant.

In particular, the probability that a patient, e.g. a patient affected with MS, who exhibits a G/G homozygosity at the SNP biallelic marker contained in SEQ ID N^{o}1 will be responsive to treatment with a INF-agent is higher than that observed for a population of patients with the same pathology but who have not the same genotype for the said biallelic marker, i.e. who do not exhibit a GIG homozygosity at the SNP biallelic marker contained in SEQ ID N^{o}1

For instance, as disclosed in the examples herein, odds for a non-response to IFNβ is decreased in patients that are homozygotes for allele G in the SNP biallelic marker contained in SEQ ID N^{o}1, i.e. the biallelic marker rs2660 contained in the OAS1 gene. On the other hand, the risk that a patient consists of a non responder is increased in patients homozygotes for allele A in the same SNP biallelic marker (see table 4 in the examples herein).

As intended herein, a patient who does not simultaneously carry a guanine at the biallelic marker contained in SEQ IDN^{o}1 in both copies of the said OAS gene of said patient, may particularly carry at least one of an adenine—at the said biallelic marker, on at least one copy of said OAS gene of said patient.

Also, the probability that a patient, e.g. a patient affected with MS, who exhibits a C/C homozygosity at the SNP biallelic marker contained in SEQ ID N^{o}2 will be non-responsive to a treatment with a INF-agent is higher than that observed for a population of patients with the same pathology but who have not the same genotype for the said biallelic marker, i.e. who do not exhibit a C/C homozygosity at the SNP biallelic marker contained in SEQ ID N^{o}2

For instance, as disclosed in the examples herein, odds for a non-response to IFNβ is increased in patients that are homozygotes for allele C in the SNP biallelic marker contained in SEQ ID N^{o}2, i.e. the biallelic marker rs1131532 contained in the TRAIL gene (see table 4 in the examples herein).

As intended herein, a patient who does not simultaneously carry a cytosine at the biallelic marker contained in SEQ IDN^{o}2 in both copies of the said TRAIL gene of said patient, may particularly carry at least one of an thymidine—at the said biallelic marker, on at least one copy of said TRAIL gene of said patient.

### Prediction Method of the Invention:

An object of the invention consists of a method for predicting the response of a patient to a medical treatment with interferon comprising the following steps:
a) genotyping a nucleic acid sample from the said patient by determining the identity of the nucleotide at the biallelic marker located at position 301 in the nucleic acid sequence of SEQ ID N^{o}1 or in the complement thereof, and
b) predicting the response of the said patient to a medical treatment with interferon, wherein:
   (i) a detection of a G/G homozygosity at the said biallelic marker location in SEQ ID N^{o}1, or detection of a C/C homozygosity at the said biallelic marker location in the complement of SEQ ID N^{o}1, is indicative of an increased risk that the said patient consists of a good responder to interferon treatment with respect to standard responsiveness, and
   (ii) a detection of a A/A homozygosity, at the said biallelic marker location in SEQ ID N^{o}1, or detection of a T/T homozygosity at the said biallelic marker location in the complement of SEQ ID N^{o}1, is indicative of an increased risk that the said patient consists of a non responder to interferon treatment with respect to standard responsiveness.

The method above is based on the identification of a particular allele at a biallelic marker, whose presence in a homozygous form within a patient's genome allows distinguishing (i) patients consisting of responders to a treatment with a IFN agent from (ii) patients consisting of non-responders to a treatment with a IFN agent, the said IFN agent consisting preferably of a type 1 IFN, including a IFNα or a IFNβ.

The identity of the variable nucleotide of the said SNP biallelic marker is determined by genotyping a nucleic acid sample previously collected from the said patient.

Generally, any source of nucleic acids, in purified or non-purified form, can be utilized as the starting nucleic acid sample, provided it contains or is suspected of containing the specific nucleic acid sequence desired. DNA or RNA may be extracted from cells, tissues, body fluids and the like.

The nucleic acid sample may be obtained from any cell source or tissue biopsy. Non-limiting examples of cell sources available include without limitation blood cells, buccal cells, epithelial cells, fibroblasts, or any cells present in a tissue obtained by biopsy. Cells may also be obtained from body fluids, such as blood or lymph, etc. DNA may be extracted using any methods known in the art, such as described in Sambrook et al., 1989.

The SNPs may be detected the nucleic acid sample, preferably after amplification. For instance, the isolated DNA may be subjected amplification by polymerase chain reaction (PCR), using oligonucleotide primers that are specific for a mutated site or that enable amplification of a region containing the mutated site. According to a first alternative, conditions for primer annealing may be chosen to ensure specific reverse transcription (where appropriate) and amplification; so that the appearance of an amplification product be a diagnostic of the presence of a particular mutation. Otherwise, DNA may be amplified, after which a mutated site may be detected in the amplified sequence by hybridization with a suitable probe or by direct sequencing, or any other appropriate method known in the art.

The PCR technology is the preferred amplification technique used in the present invention. A variety of PCR techniques are familiar to those skilled in the art. For a review of PCR technology, see Molecular Cloning to Genetic Engineering White, B. A. Ed. in Methods in Molecular Biology 67: Humana Press, Totowa (1997) and the publication entitled “PCR Methods and Applications” (1991, Cold Spring Harbor Laboratory Press). In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195, 4,683,202 and 4,965,188.

The identification of biallelic markers as described herein allows the design of appropriate oligonucleotides, which can be used as primers to amplify DNA fragments comprising the biallelic markers of the present invention. Amplification can be performed using the primers initially used to discover new biallelic markers which are described herein or any set of primers allowing the amplification of a DNA fragment comprising a biallelic marker of the present invention. Primers can be prepared by any suitable method. As for example, direct chemical synthesis by a method such as the phosphodiester method of Narang S. A. et al. (Methods Enzymol. 68:90-98, 1979), the phosphodiester method of Brown E. L. et al. (Methods Enzymol. 68:109-151, 1979), the diethylphosphoramidite method of Beaucage et al. (Tetrahedron Lett. 22:1859-1862, 1981) and the solid support method described in EP 0 707 592.

Actually numerous strategies for genotype analysis are available. Briefly, the nucleic acid molecule may be tested for the presence or absence of a restriction site. When a base substitution mutation creates or abolishes the recognition site of a restriction enzyme, this allows a simple direct PCR test for the mutation. Further strategies include, but are not limited to, direct sequencing, restriction fragment length polymorphism (RFLP) analysis; hybridization with allele-specific oligonucleotides (ASO) that are short synthetic probes which hybridize only to a perfectly matched sequence under suitably stringent hybridization conditions; allele-specific PCR; PCR using mutagenic primers; ligase-PCR, HOT cleavage; denaturing gradient gel electrophoresis (DGGE), temperature denaturing gradient gel electrophoresis (TGGE), single-stranded conformational polymorphism (SSCP) and denaturing high performance liquid chromatography. Direct sequencing may be accomplished by any method, including without limitation chemical sequencing, using the Maxam-Gilbert method; by enzymatic sequencing, using the Sanger method; mass spectrometry sequencing; sequencing using a chip-based technology; and real-time quantitative PCR. Preferably, DNA from a subject is first subjected to amplification by polymerase chain reaction (PCR) using specific amplification primers. However several other methods are available, allowing DNA to be studied independently of PCR, such as the rolling circle amplification (RCA), the Invader®assay, or oligonucleotide ligation assay (OLA). OLA may be used for revealing base substitution mutations. According to this method, two oligonucleotides are constructed that hybridize to adjacent sequences in the target nucleic acid, with the join sited at the position of the mutation. DNA ligase will covalently join the two oligonucleotides only if they are perfectly hybridized.

The SNPs of the invention may be identified by using DNA chip technologies as those described in documents WO 2004/106546 and WO 2006/001627.

In certain embodiments of the prediction method above, step a) of genotyping may be performed by a technique of Allele Specific Amplification. According to this technique, discrimination between the two alleles of a biallelic marker can be achieved by a selective strategy, whereby one of the alleles is amplified without amplification of the other allele. This is accomplished by placing a polymorphic base at the 3′ end of one of the amplification primers. Because the extension forms from the 3′end of the primer, a mismatch at or near this position has an inhibitory effect on amplification. Therefore, under appropriate amplification conditions, these primers only direct amplification on their complementary allele. Designing the appropriate allele-specific primer and the corresponding assay conditions are well with the ordinary skill in the art.

Nucleic acids having at least 10 nucleotides and exhibiting sequence complementarity or homology to the sequence of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, and enzymatic or other ligands (e. g. avidin/biotin). Probes typically comprise single-stranded nucleic acids of between 10 to 1000 nucleotides in length, for instance of between 10 and 800, more preferably of between 15 and 700, typically of between 20 and 500.

Any of the polynucleotides, primers and probes that may be used for performing the prediction method of the invention can be conveniently immobilized on a solid support. Solid supports are known to those skilled in the art and include the walls of wells of a reaction tray, test tubes, polystyrene beads, magnetic beads, nitrocellulose strips, membranes, microparticles such as latex particles, sheep (or other animal) red blood cells, duracytes.®. and others. The solid support is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and duracytes are all suitable examples. Suitable methods for immobilizing nucleic acids on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, duracytes®. and other configurations known to those of ordinary skill in the art. The polynucleotides of the invention can be attached to or immobilized on a solid support individually or in groups of at least 2, 5, 8, 10, 12, 15, 20, or 25 distinct polynucleotides to a single solid support.

Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

According to another aspect of the invention, the mutation of interest is detected by contacting the nucleic sample of the patient with a nucleic acid probe, which is optionally labeled. Primers may also be useful to amplify or sequence the portion of the OAS1 gene (e.g. SEQ ID NO:1) containing the mutated positions of interest.

Such probes or primers are nucleic acids that are capable of specifically hybridizing with a portion of the OAS1 gene sequence (e.g. SEQ ID NO: 1) containing the mutated positions of interest. That means that they are sequences that hybridize with the portion mutated OAS1 nucleic acid sequence to which they relate under conditions of high stringency.

Oligonucleotide probes or primers may contain at least 10, 15, 20 or 30 nucleotides. Their length may be shorter than 400, 300, 200 or 100 nucleotides.

In certain preferred embodiments,the primers are selected to be substantially complementary to the different strands of each specific sequence to be amplified. The length of the primers of the present invention can range from 8 to 100 nucleotides, preferably from 8 to 50, 8 to 30 or more preferably 8 to 25 nucleotides. Shorter primers tend to lack specificity for a target nucleic acid sequence and generally require cooler temperatures to form sufficiently stable hybrid complexes with the template. Longer primers are expensive to produce and can sometimes self-hybridize to form hairpin structures. The formation of stable hybrids depends on the melting temperature (Tm) of the DNA. The Tm depends on the length of the primer, the ionic strength of the solution and the G+C content. The higher the G+C content of the primer, the higher is the melting temperature because G:C pairs are held by three H bonds whereas A:T pairs have only two. The G+C content of the amplification primers of the present invention preferably ranges between 10 and 75%, more preferably between 35 and 60%, and most preferably between 40 and 55%. The appropriate length for primers under a particular set of assay conditions may be empirically determined by one of skill in the art.

The spacing of the primers determines the length of the segment to be amplified. In the context of the present invention amplified segments carrying biallelic markers can range in size from at least about 25 bp to 35 kbp. Amplification fragments from 25-3000 bp are typical, fragments from 50-1000 bp are preferred and fragments from 100-600 bp are highly preferred. It will be appreciated that amplification primers for the biallelic markers may be any sequence which allow the specific amplification of any DNA fragment carrying the markers. Amplification primers may be labeled or immobilized on a solid support, according to techniques well known from the one skilled in the art.

The prediction method according to the invention is preferably performed on nucleic acid samples originating from patients who are affected with a disease selected from the group consisting of Multiple Sclerosis, hepatitis or a cancer.

The prediction method according to the present invention is preferably performed on nucleic acid samples from patients that are affected with a disease that may be prevented or treated with a recombinant human type 1 interferon, including IFNα and IFNβ. In preferred embodiments, the prediction method according to the invention is performed on nucleic acid samples originating from patients that are affected with a disease that may be prevented or treated with a recombinant human IFNβ-1a or with a recombinant human IFNβ-1b.

Typically, the prediction method of the invention is performed on nucleic acid samples originating from patients that are affected with Multiple Sclerosis, which disease may be prevented or treated with a recombinant human IFNβ, including recombinant human IFNβ-1a and recombinant human IFNβ-1b.

As already described previously herein, the inventors have found that there is a statistically significant relationship between the allele identity of the biallelic marker located at position 301 of SEQ ID N^{o}2, which is contained in the TRAIL gene sequence, and the kind of response of a patient to a treatment with an IFN agent.

It has been shown in the examples herein that the probability that a patient, e.g. a patient affected with MS, who exhibits a C/C homozygosity at the SNP biallelic marker contained in SEQ ID N^{o}2 will be non-responsive to a treatment with a INF-agent is higher than that observed for a population of patients with the same pathology but who have not the same genotype for the said biallelic marker, i.e. who do not exhibit a C/C homozygosity at the SNP biallelic marker contained in SEQ ID N^{o}2.

Conversely, the probability that a patient, e.g. a patient affected with MS, who exhibits a T/T homozygosity at the SNP biallelic marker contained in SEQ ID N^{o}2 will be responsive to a treatment with a INF-agent is higher than that observed for a population of patients with the same pathology but who have not the same genotype for the said biallelic marker, i.e. who do not exhibit a T/T homozygosity at the SNP biallelic marker contained in SEQ ID N^{o}2.

Thus, the biallelic marker above that is contained in SEQ ID N^{o}2 may be used in combination with the biallelic marker contained in SEQ ID N^{o}1 for performing the prediction method according to the invention.

Consequently, in certain embodiments of the prediction method of the invention, step a) further comprises determining the identity of the nucleotide at the biallelic marker located at position 301 in the nucleic acid sequence of SEQ ID N^{o}2 and wherein at step b):
(i) a detection of a T/T homozygosity at the said biallelic marker location in SEQ ID N^{o}2, or of a A/A homozygosity at the said biallelic marker location in the complement of SEQ ID N^{o}2, is indicative of an increased risk that the said patient consists of a good responder to interferon treatment with respect to standard responsiveness, and
(ii) a detection of a C/C homozygosity at the said biallelic marker location in SEQ ID N^{o}2, or of a G/G homozygosity at the said biallelic marker location in the complement SEQ ID N^{o}2, is indicative of an increased risk that the said patient consists of a non responder to interferon treatment with respect to standard responsiveness.

According to the embodiment above of the prediction method according to the invention, it is made use of a combination of two biallelic markers that define specific marker haplotypes which enable a further increased statistical relevance of the method for predicting the response of the patient tested to a medical treatment by an IFN agent, typically to a type 1 IFN agent, preferably IFNα or IFNβ, which include IFNβ-1a and IFNβ-1b.

Indeed, when performing the prediction method according to the invention, the most relevant prediction result that the tested patient consists of a good responder to a treatment with an IFN agent is obtained when :
a G/G homozygosity is found at the biallelic marker contained in SEQ ID N^{o}1, or a C/C homozygosity is found at the biallelic marker contained in the complement of SEQ ID N^{o}1 and
no C/C homozygosity is found at the biallelic marker contained in SEQ ID N^{o}2, or no G/G homozygosity is found at the biallelic marker contained in the complement of SEQ ID N^{o}2.

Also, when performing the prediction method according to the invention, the most relevant prediction result that the tested patient consists of a non-responder to a treatment with an IFN agent is obtained when:
no G/G homozygosity is found at the biallelic marker contained in SEQ ID N^{o}1, or no C/C homozygosity is found at the biallelic marker contained in the complement of SEQ ID N^{o}1, and
a C/C homozygosity is found at the biallelic marker contained in SEQ ID N^{o}2, or a G/G homozygosity is found at the biallelic marker contained in the complement of SEQ ID N^{o}2.

Thus, in the above embodiment of the prediction method of the invention, step a) consists of genotyping the nucleic acid sample from the patient under test by determining the identity of the variable nucleotide located at the biallelic marker location of each of the two biallelic markers contained in SEQ ID N^{o}1 and in SEQ ID N^{o}2, respectively.

### IFN-Agents:

As indicated previously herein, IFN agents include recombinant human IFN type I. Alpha and beta interferons, which are grouped together as type I interferon, are produced by white blood cells and a type of connective tissue cell called a fibroblast.

The alpha and beta interferons share some biological activities, but also have activities that are distinct from one another. These similarities and differences reflect the common and different binding of the interferons to various targets (receptors) on the surfaces of human cells. Interferon alpha and interferon beta bind to a common receptor sometimes known as the IFN alpha/betaR made up of two subunits (IFNAR1 and IFNAR2). Naturally occurring wild-type IFNAR1 protein is a 557-amino acid protein and naturally occurring human wild-type IFNAR2 protein is a 515-amino acid protein

Alpha interferon is manufactured by Roche Products (trade name Pegasys) and Schering-Plough (Viraferon-Peg). Biogen (Avonex) and Serono (Rebif) both market an interferon-designated beta-1a. Both of the beta-1a interferons are produced in genetically engineered mammals. For example, Rebif is produced in Chinese hamster ovary cells that contain the gene coding for human interferon beta.

An interferon designated as beta-1b enhances the activity of T-cells, while simultaneously reducing the production cytokines that operate in the inflammatory response to infection and injury. As well, this interferon retards the exposure of antigens on the surface of cells (and so lessens the development of an immune response to the antigens), and retards the appearance of white blood cells (lymphocytes) in the central nervous system.

The reduction of the immune response can lessen the damage to nerve cells in diseases such as multiple sclerosis. In this disease, the immune system is stimulated to react against the myelin sheath that surrounds the cells, a phenomenon called demyelination. Demyelination produces a malfunction in the transmission of impulses from nerve to nerve and from nerve to muscle.

Infection with the virus that causes hepatitis C is hindered by interferon via the binding to a site on human cells that is also used by the virus. Thus, the virus cannot enter and infect the host cell.

Interferons are normally administered by subcutaneous or intramuscular injection. These active ingredients are not administered orally because the strong digestive enzymes of the stomach would degrade them.

For use in multiple sclerosis, interferon beta-1a is injected into the muscle (intramuscular injection), and beta-1b is injected just below the skin (subcutaneous injection). The injections are usually given every other day. The recommended dose for beta-1a and 1 b is 0.03 mg and 0.25 mg, respectively. Initial doses of beta-1b should be far less (i.e., 0.0625 mg), with a gradual increase in dose over six weeks.

Diseases Treated with an Interferon Agent:

The diseases treated with an Interferon agent include Multiple Sclerosis, hepatitis, cancer, chronic inflammatory demyelinating polyradiculoneuropathy, hypereosinophilic syndromes, human T-cell lymphotropic virus type 1-associated myelopathy and Behcet's disease. In one embodiment of a prediction method according to the invention, the disease is selected from the group consisting of multiple myeloma, hepatocarcinoma, renal cancer, melanoma, chronic myeloid leukemia]. In another embodiment, the disease is selected from the group consisting of Hepatitis B and C.

In a most preferred embodiment, the disease treated with Interferon agent is Multiple Sclerosis Disorder.

The prediction method of the invention is particularly useful to predict the response to a treatment by an IFN agent in a patient who is affected with Multiple Sclerosis.

After being tested for responsiveness to a treatment with IFN agent using the prediction method described herein, the patients that have been predicted to consist of good responders may be administered with an IFN agent with a good expectation of success of the medical treatment.

### Kits:

The present invention further provides kits suitable for determining the haplotype of the invention.

The kits may include the following components:
(i) a probe, usually made of DNA, and that may be pre-labelled. Alternatively, the probe may be unlabelled and the ingredients for labelling may be included in the kit in separate containers; and
(ii) hybridization reagents: the kit may also contain other suitably packaged reagents and materials needed for the particular hybridization protocol, including solid-phase matrices, if applicable, and standards.

In another embodiment, the kits may include:
(i) sequence determination or amplification primers: sequencing primers may be pre-labelled or may contain an affinity purification or attachment moiety; and
(ii) sequence determination or amplification reagents: the kit may also contain other suitably packaged reagents and materials needed for the particular sequencing amplification protocol. In one preferred embodiment, the kit comprises a panel of sequencing or amplification primers, whose sequences correspond to sequences adjacent to at least one of the polymorphic positions, as well as a means for detecting the presence of each polymorphic sequence.

In a particular embodiment, it is provided a kit which comprises one or more nucleotide primers specific for amplifying a nucleic acid contained in the OAS1 gene and wherein the said nucleic acid comprises the variable nucleotide of the biallelic marker contained in SEQ ID N^{o}1. Another kit can additionally comprise a pair of nucleotide primers specific for amplifying a nucleic acid contained in the TRAIL gene and wherein the said nucleic acid comprises the variable nucleotide of the biallelic marker contained in SEQ ID N^{o}2.

### Methods of Treatment

Another object of the invention consists of a method for the treatment of a patient who is affected with a disease that may be prevented or treated with an IFN agent, wherein the said method comprises the steps of:
a) predicting if the said patient consists of a good responder or of a non-responder to a treatment with an IFN agent, by performing the prediction method that is described above in the present specification; and
b) administering an IFN agent to the said patient when the said patient has been predicted, at step a), to consist of a good responder to a treatment with an IFN agent.

Preferably, the said patient is affected with a disease selected from the group consisting of Multiple Sclerosis, hepatitis or a cancer. Most preferably, the said patient is affected with Multiple Sclerosis.

Advantageously, the IFN agent consists of a type 1 IFN agent, preferably IFNα or IFNβ, which include IFNβ-1a and IFNβ-1b.

Details relating to the administration regimen of an IFN agent have been previously described in the present specification.

In some embodiments of step a) of the method of treatment above, step a) consists of genotyping the nucleic acid sample from the patient by determining the identity of the variable nucleotide located at the biallelic marker location the biallelic marker contained in SEQ ID N^{o}1.

In some other embodiments of step a) of the method of treatment above, step a) consists of genotyping the nucleic acid sample from the patient by determining the identity of the variable nucleotide located at the biallelic marker location of each of the two biallelic markers contained in SEQ ID N^{o}1 and in SEQ ID N^{o}2, respectively.

This invention also relates to the use of an IFN agent for manufacturing a medicament for treating a disease in a patient in need thereof, and wherein the said patient is selected from one of the following:
(i) a patient, the genome of which exhibits a G/G homozygosity at the said biallelic marker location in SEQ ID N^{o}1, or the genome of which exhibits a C/C homozygosity at the said biallelic marker location in the complement of SEQ ID N^{o}1, and
(ii) a patient, the genome of which (1) exhibits a G/G homozygosity at the said biallelic marker location in SEQ ID N^{o}1, or the genome of which exhibits a C/C homozygosity at the said biallelic marker location in the complement of SEQ ID N^{o}1, and which (2) further exhibits no C/C homozygosity at the said biallelic marker location in SEQ ID N^{o}2, or of no G/G homozygosity at the said biallelic marker location in the complement SEQ ID N^{o}2

Preferably, the said patient is affected with a disease selected from the group consisting of Multiple Sclerosis, hepatitis or a cancer. Most preferably, the said patient is affected with Multiple Sclerosis.

Advantageously, the IFN agent consists of a type 1 IFN agent, preferably IFNα or IFNβ, which include IFNβ-1a and IFNβ-1b.

This invention is further illustrated in the examples hereafter.

### Example

### A. Materials and Methods

### A.1. Subjects and Response to IFNβ Treatment Definition

Patients naïve to immunotherapy and starting IFNβ treatment during the period January 1995 to December 1998 were recruited at the Neuroimmunology Unit, Hospital vall d'hebron, Barcelona (Villoslada et al., 2002), Spain; the Department of Neurology, University of Navarra, Pamplona, Spain and the MS Centre, Toulouse, France, using identical inclusion criteria, which required a diagnosis of definite MS according to Poser criteria (Poser et al., 1983), age between 18 and 50 years old, and a history of two or more relapses in the previous 2 years. Exclusion criteria included pregnancy, inability to give informed consent, dementia, cancer or recreational drug abuse. Patients were followed-up prospectively at least for 2 years since initiation of therapy. Clinical data such as the EDSS was recorded every 3 months. Further, for each patient we recorded the sex, age at disease onset, disease type, disease duration and EDSS at the beginning of the treatment, number of relapses 2 years before the treatment, as well as EDSS and number of relapses during the first two years of treatment. Finally, the progression index (a ratio between EDSS and disease duration) was calculated at treatment onset. MRI was performed at the time of diagnosis but was not used to monitor the treatment. All known ancestors were European in origin.

The primary end-point of treatment response was the suppression of relapses during follow-up (exacerbation-free patients) and no increase in the EDSS confirmed by two consecutive visits. Using the primary end point, we categorized patients as follows: responders were defined as having no relapses and no increase of the EDSS after a 2 years follow-up period. Non-responders were defined as having suffered two or more relapses or having an increase of 1 point in the EDSS confirmed in two consecutive visits in the 2 years follow-up period. Patients with an intermediate response (experiencing 1 relapse or 0.5 increase in the EDSS during the study) were excluded in order to minimise the risk of misclassification. Relapses require the appearance of a new symptom or worsening of an old symptom that could be attributed to MS activity, in the absence of fever over at least 24 h, preceded by stability or improvement for at least 30 days. Relapses were confirmed by neurological examination. All studies were approved by the respective committees of human research. Informed consent was obtained for all study participants.

### A2. Candidate Genes, SNP Selection, and Genotyping

Thirteen SNPs in eight ISG genes were selected as candidates for this study. Details of the polymorphisms are shown in Table 2. SNP genotyping was performed in an Applied Biosystem 9700 thermal cycler with TaqMan probe-based, 5′ nuclease assays purchased from ABI. Genomic DNA samples (10 μg/μl) are organized into genotyping keys and 5 μl aliquoted into 394 well plates for PCR amplification. Preliminary experiments establish optimal PCR conditions for each primer pair. All experiments include controls with genotypes confirmed by sequencing. Generation of genotypes is performed blind to the clinical status of the subjects. Allele information is then exported into Excel and saved for direct downloading into our data management system for analysis and storage.

TABLE 2
Genes and polymorphisms

| Gene | SNP | Variation | Reported pharmacogenomic effect |
|---|---|---|---|
| CTLA4 | −318 | C/T | Sustained response to IFNα + |
| | promoter | | ribavirin in Hepatitis C |
| | rs231775 | A/G | No previous report |
| IL-10 | −1082 | A/G | Sustained response to IFNα in |
| | promoter | | Hepatitis C |
| | −819 | T/C | Sustained response to IFNα in |
| | promoter | | Hepatitis C |
| IRF1 | −300 | G/A | Sustained response to IFNβ in |
| | promoter | | Hepatitis C |
| | rs839 | A/G | No previous report |
| IRF 4 | rs1050972 | C/G | Transcription-based prediction of |
| | | | response to IFNbeta in MS |
| MX1 | rs2070229 | T/C | Role in self limiting HCV infection |
| | rs469390 | G/A | No previous report |
| OAS1 | rs 2660 | A/G | Role in self limiting HCV infection |
| MAP3K3 | rs3733951 | A/T | Transcription-based prediction of |
| | | | response to IFNbeta in MS |
| | rs702689 | A/G | Transcription-based prediction of |
| | | | response to IFNbeta in MS |
| TRAIL | rs1131532 | C/T | Transcription-based prediction of |
| | | | response to IFNbeta in MS |

### A3. Statistical Analysis

Demographic and clinical data were analysed according to the response to IFNβtreatment. For variables with a normal distribution, descriptive results are presented as the mean±SE. The t test for unpaired samples was used to analyse the differences between the two groups (responders/non responders). For variables with a skewed distribution, results are presented as the median and the interquartile range. The Mann-Whitney U test was used to analyse the differences between the two groups Qualitative variables were compared using the Chi-square test or Fisher's exact test according to the size of the series.

To look for associations between genes polymorphisms and clinical outcome (non responder to treatment), a multiple logistic regression was performed. The individual alleles were compared and tabulated under Genotype 1 (binary variable where 11 and 12 are compared to 22; so allele 1+ vs. allele 1−) and Genotype 2 (binary variable where 12 and 22 are compared to 11; so allele 2+ vs. allele 2−). One model was used for each gene polymorphism. Sex, disease type, disease duration at treatment onset, interferon type, and number of relapse before treatment onset were included in each model as they were potential confounders. In a secondary analysis, we looked at a potential interaction between genes associated with an increase risk for non-response (IRNR) to IFN□ treatment. All statistical tests were two-tailed and a p<0.05 was considered as significant. Data analysis was performed by using Stata 7.0 software (Stata Corp, College Station, TX).

### Example 1

### Identification of Polymorphysms Associated for Prediction of the Response to Interferon Treatment

Ninety patients met the criteria for positive response to IFNβ treatment and ninety for non-response. Patients with intermediate response were excluded (81 patients). No differences were observed between responders and non responders for the following variables: sex, mean age at disease onset, EDSS, disease duration, number of relapses and progression index at treatment onset, irrespective of the recruiting Centre (See table 3). As expected, the proportion of patients with a secondary progressive disease was higher in the non responder group (p=0.018).

TABLE 3

| | | Responder | | Non Responder | | |
|---|---|---|---|---|---|---|
| | | n = 90 | | n = 90 | | P value |
| Origin; n (%) | Toulouse | 10 | (11.1) | 14 | (15.6) | NS |
| | Pamplona | 5 | (5.6) | 7 | (7.8) | |
| | Barcelona | 75 | (83.3) | 69 | (76.7) | |
| Sex; n (%) | F | 64 | (71.9) | 56 | (62.2) | NS |
| | M | 25 | (28.1) | 34 | (37.8) | |
| Age at onset; mean (±SD) | | 25.9 | (7.6) | 25.1 | (7.5) | NS |
| MS Type; n (%) | RR | 79 | (97.5) | 73 | (86.9) | 0.018 |
| | SP | 2 | (2.5) | 11 | (13.1) | |
| Age at treatment onset; | | 31 | (27-37) | 31.5 | (24-37) | NS |
| med(iqr*) | | | | | | |
| EDSS at treatment onset; n | <3 | 69 | (76.7) | 57 | (63.3) | NS |
| (%) | | | | | | |
| | ≧3 | 21 | (23.3) | 33 | (36.7) | |
| Disease duration at treatment | | 4.5 | (2-7) | 4 | (2-8) | NS |
| onset; med(iqr*) | | | | | | |
| Progression Index at | | 0.5 | (0.2-0.8) | 0.5 | (0.3-1.2) | NS |
| treatment onset; med (iqr*) | | | | | | |
| Number of relapses 2 years | | 2 | (2-3) | 3 | (2-4) | NS |
| before Treatment; med (iqr*) | | | | | | |
| Treatment; n (%) | Betaferon | 46 | (56.8) | 43 | (51.2) | 0.004 |
| | Avonex | 26 | (32.1) | 15 | (17.9) | |
| | Rebif | 9 | (11.1) | 26 | (31.0) | |

Odds for a non-response to IFNβ is decreased in patients homozygote for allele G in SNP rs2660 of the OAS1 gene (OR=0.3; p=0.038; 95% Cl=0.1-0.9). On the other hand, the risk to be a non responder is increased in patients homozygote for allele A in the same OAS1 SNP (OR=2.6, p=0.009; 95% Cl=1.3-5.2) and for allele C in SNP rs1131532 of the TRAIL gene (OR=2.1, p=0.041; 95% Cl=1.0-4.2) (see table 4).

TABLE 4
Multivariate analysis

| Genes | Genotype¹ | Odds Ratio² | [95% CI³] | P. value |
|---|---|---|---|---|
| IL10 | X1 | 1.2 | 0.6-2.3 | 0.628 |
| | X1r | 1.2 | 0.4-3.6 | 0.729 |
| IL10_2 | X2 | 2.4 | 0.9-3.4 | 0.081 |
| | X2r | 0.7 | 0.3-1.4 | 0.312 |
| CTLA4-1 | X3 | 0.4 | 0.1-1.4 | 0.151 |
| | X3r | 1.5 | 0.7-3.0 | 0.258 |
| CTLA4-2 | X4 | No patient | | |
| | X4r | 0.9 | 0.3-2.2 | 0.802 |
| IRF1 | X5 | 0.9 | 0.5-1.9 | 0.902 |
| | X5r | 1.0 | 0.3-2.9 | 0.976 |
| IRF1-2 | X6 | 1.1 | 0.3-3.2 | 0.898 |
| | X6r | 0.9 | 0.5-1.9 | 0.870 |
| IRF4 | X7 | No patient | | |
| | X7r | 0.3 | 0.1-1.1 | 0.064 |
| MAP3K3 | X8 | No patient | | |
| | X8r | 0.6 | 0.2-1.8 | 0.420 |
| MAP3K3- | X9 | 2.5 | 0.8-8.3 | 0.124 |
| | X9r | 0.6 | 0.3-1.2 | 0.136 |
| MX1 | X10 | 1.1 | 0.5-2.6 | 0.762 |
| | x10r | 2.2 | 1.0-5.1 | 0.059 |
| OAS1 rs2660 | x11 (G/G) | 0.3 | 0.1-0.9 | 0.038 |
| | x11r (A/A) | 2.6 | 1.3-5.2 | 0.009 |
| MX1-2 | X12 | 1.0 | 0.5-2.1 | 0.925 |
| | x12r | 1.6 | 0.6-4.1 | 0.335 |
| TRAIL rs1131532 | x13 (C/C) | 2.1 | 1.0-4.2 | 0.041 |
| | X13r | 0.5 | 0.1-2.1 | 0.350 |

¹For each SNP (allele variant 1 or 2) Genotypes have been modelised as follow: X (binary variable where 11 and 12 are compared to 22; so allele 1+ vs. allele 1−) and Genotype Xr (binary variable where 12 and 22 are compared to 11; so allele 2+ vs. allele 2−). Overall 26 multivariate analysis have been performed
²Adjusted for sex. MS type. number of relapses after 2 years of treatment. EDSS at treatment onset. disease duration at treatment onset. interferon type.
³CI. confidence interval

In this study the inventors report one gene, OAS1, carrying polymorphism associated with failure response to IFNβ therapy in a well-characterized MS dataset from the French-Spanish border at both sides of the Pyrenees (Barcelona, Pamplona, and Toulouse), suggesting their involvement in the therapeutic effect and their potential value as pharmacogenenomic markers.

OAS1 maps to 12q24.1 and encodes for a key enzyme in the IFNα and β regulation cascade, the 2′5′ oligoadenylate synthetase. Briefly, after their binding to the IFNAR receptor and triggering of the JAK/STAT cascade, there is an activation of transcriptional factors that will interact with a promoter sequence called interferon stimulated response element (ISRE) (Darnell et al., 1994). A polymorphism in the 3′-untranslated region of the OAS1 gene was reported to be associated with the outcome of hepatitis C virus infection (Knapp et al., 2003), whereas a nonsynonymous A/G SNP in exon 3 appears to affect susceptibility to SARS (Hamano et al., 2005). Further, OAS1 has also been implicated in autoimmune disease such as type I diabetes (Field et al., 2005; Bonnevie-Nelsen et al., 2000). More recently, increased enzymatic activity was shown to be directly associated with OAS1 genotypes. The three genotypes, AA, GA, and GG showed systematic variation in basal 2′5′ oligoadenylate synthetase antiviral activity and intrafamilial variation linked to genetic distance; (Bonnevie-Nelsen et al., 2005). Patients carrying the GG and GA variant at OAS1-rs2660 had elevated oligoadenylate synthetase activity when compare to AA carriers. In the MS dataset, rs2660 G/G homozygotes cluster primarily within the IFNβ good responders group, whereas MS patients A/A homozygote for the same SNP (non G carrier) have an increase risk to be non responders (OR=2.6, p=0.009). Hence higher enzyme activity mediated by the rs2660*G allele is associated with a favourable response to IFNβ treatment. Although 2′5′ oligoadenylate synthetase has a key role in RNA viral degradation and clearance through activation of a latent ribonuclease (RNAseL), the pharmacogenomic effect is most likely associated with the apoptotic function of the RNAseL system. (Bonnevie-Nelsen et al., 2005) TRAIL maps to 3q26 and encodes a cytokine that belongs to the tumour necrosis factor (TNF) ligand family. This protein preferentially induces apoptosis in transformed and tumour cells. TRAIL has been implicated in autoimmune diseases such as MS. It appears that TRAIL may be a paradigm of neuroinflammation pathogenesis complexity. In one hand, it has been postulated that TRAIL was a major player leading to central neural damage in the mouse model of MS (Aktas et al., 2005). In the other hand, TRAIL may have an immunomodulatory function since mice lacking TRAIL experienced an increased susceptibility to autoimmunity (Lamhamedi et al., 2003). The mechanism could be an inhibition of autoreactive T cells activation²³, probably involving thymocyte apoptosis(Lamhamedi et al., 2003). Nevertheless, in humans, TRAIL does not appear to induce apoptosis of antigen specific T cells, rather TRAIL may inhibit T cell activation. This could be linked to a blockade of calcium influx. (Lunemann et al., 2002) The potential relevance of TRAIL as a biological marker for predicting response to IFNβ was previously suggested by a study reporting that patients with high concentration of soluble TRAIL in the serum have a better response to—therapy (Wandinger et al., 2003). In addition, a gene expression study of peripheral blood mononuclear cell (PBMC) demonstrated that responders experienced an early and sustained induction of TRAIL (Wandinger et al., 2003). Here we found that patients C/C homozygote for the SNP rs1131532, had an increased risk to be non responders as compare to C/T and T/T patients (OR=2.1, p=0.041). This SNP is located in a coding region and is responsible of a synonymous variation.

All together, these results are consistent with previously proposed apoptosis-mediated therapeutic mechanisms of IFNs in both, cancer and MS (Chawla-Sarkar, 2003; Gniadek et al., 2003). Specifically, activation of programmed cell death could lead to a reduction in the number of activated lymphocytes, macrophages and monocyte-derived dendritic cells, all key components of the pathogenic process leading to tissue damage in MS (Van Weyenbergh et al., 2001; Kayagaki et al., 1999; Lehner at al., 2001). This is consistent with Baranzini et al. reporting a transcriptional patterns in genes mediating apoptosis associated with poor response to IFNβ therapy (Baranzini et al., 2005). This study suggests that genetic variants in OAS1 and TRAIL genes may be of clinical interest in MS as predictors of the response to IFNβ therapy.

### REFERENCES

Darnell J E, Jr., Kerr I M, Stark G R. Jak-STAT pathways and transcriptional activation in response to IFNs and other extracellular signaling proteins. Science 1994; 264(5164):1415-21.
Knapp S, Yee L J, Frodsham A J, et al. Polymorphisms in interferon-induced genes and the outcome of hepatitis C virus infection: roles of M×A, OAS-1 and PKR. Genes Immun 2003; 4(6):411-9.
Baranzini S E, Mousavi P, Rio J, et al. Transcription-based prediction of response to IFNbeta using supervised computational methods. PLoS Biol 2005; 3(1):e2.
Wandinger K P, Lunemann J D, Wengert O, et al. TNF-related apoptosis inducing ligand (TRAIL) as a potential response marker for interferon-beta treatment in multiple sclerosis. Lancet 2003; 361(9374):2036-43.
Villoslada P, Barcellos L F, Rio J, et al. The HLA locus and multiple sclerosis in Spain. Role in disease susceptibility, clinical course and response to interferon-beta. J Neuroimmunol 2002; 130(1-2):194-201.
Poser C M, Paty D W, Scheinberg L, et al. New diagnostic criteria for multiple sclerosis: guidelines for research protocols. Ann Neurol 1983; 13(3):227-31.
Hamano E, Hijikata M, Itoyama S, et al. Polymorphisms of interferon-inducible genes OAS-1 and M×A associated with SARS in the Vietnamese population. Biochem Biophys Res Commun 2005; 329(4):1234-9.
Field L L, Bonnevie-Nielsen V, Pociot F, Lu S, Nielsen T B, Beck-Nielsen H. OAS1 splice site polymorphism controlling antiviral enzyme activity influences susceptibility to type 1 diabetes. Diabetes 2005; 54(5):1588-91.
Bonnevie-Nielsen V, Martensen P M, Justesen J, et al. The antiviral 2′,5′-oligoadenylate synthetase is persistently activated in type 1 diabetes. Clin Immunol 2000; 96(1):11-8.
Bonnevie-Nielsen V, Field L L, Lu S, et al. Variation in antiviral 2′,5′-oligoadenylate synthetase (2′5′AS) enzyme activity is controlled by a single-nucleotide polymorphism at a splice-acceptor site in the OAS1 gene. Am J Hum Genet 2005; 76(4):623-33.
Aktas O, Smorodchenko A, Brocke S, et al. Neuronal damage in autoimmune neuroinflammation mediated by the death ligand TRAIL. Neuron 2005; 46(3):421-32.
Lamhamedi-Cherradi S E, Zheng S J, Maguschak K A, Peschon J, Chen Y H. Defective thymocyte apoptosis and accelerated autoimmune diseases in TRAIL−/− mice. Nat Immunol 2003; 4(3):255-60.
Lunemann J D, Waiczies S, Ehrlich S, et al. Death ligand TRAIL induces no apoptosis but inhibits activation of human (auto) antigen-specific T cells. J Immunol 2002; 168(10):4881-8.
Chawla-Sarkar M, Lindner D J, Liu Y F, et al. Apoptosis and interferons: role of interferon-stimulated genes as mediators of apoptosis. Apoptosis 2003; 8(3):237-49.
Gniadek P, Aktas O, Wandinger K P, et al. Systemic IFN-beta treatment induces apoptosis of peripheral immune cells in MS patients. J Neuroimmunol 2003; 137(1-2):187-96.
Van Weyenbergh J, Wietzerbin J, Rouillard D, Barral-Netto M, Liblau R. Treatment of multiple sclerosis patients with interferon-beta primes monocyte-derived macrophages for apoptotic cell death. J Leukoc Biol 2001; 70(5):745-8.
Kayagaki N, Yamaguchi N, Nakayama M, Eto H, Okumura K, Yagita H. Type I interferons (IFNs) regulate tumor necrosis factor-related apoptosis-inducing ligand (TRAIL) expression on human T cells: A novel mechanism for the antitumor effects of type I IFNs. J Exp Med 1999; 189(9):1451-60.
Lehner M, Felzmann T, Clodi K, Holter W. Type I interferons in combination with bacterial stimuli induce apoptosis of monocyte-derived dendritic cells. Blood 2001; 98(3):736-42.

1-9. (canceled)

10. A method for predicting the response of a patient affected with Multiple Sclerosis disorder, hepatitis or cancer, to a medical treatment with a recombinant human type I IFNβ interferon, comprising the following steps:
a) genotyping a nucleic acid sample from said patient by determining the identity of the nucleotide at the biallelic marker located at position 301 in the nucleic acid sequence of SEQ ID N^{o}1 or in the complement thereof, and
b) predicting the response of said patient to a medical treatment with interferon, wherein:
(i) a detection of a G/G homozygosity at said biallelic marker location in SEQ ID N^{o}1, or detection of a C/C homozygosity at said biallelic marker location in the complement of SEQ ID N^{o}1, is indicative of an increased risk that said patient consists of a good responder to interferon treatment with respect to standard responsiveness, and
(ii) a detection of a A/A homozygosity at said biallelic marker location in SEQ ID N^{o}1, or detection of a T/T homozygosity at said biallelic marker location in the complement of SEQ ID N^{o}1, is indicative of an increased risk that said patient consists of a non responder to interferon treatment with respect to standard responsiveness.

11. The method according to claim 10, wherein the patient is affected with Multiple Sclerosis disorder.

12. The method according to claim 10, wherein, step a) further comprises determining the identity of the nucleotide at the biallelic marker located at position 301 in the nucleic acid sequence of SEQ ID N^{o}2, and at step b):
(i) a detection of a T/T homozygosity at said biallelic marker location in SEQ ID N^{o}2, or detection of a A/A homozygosity at said biallelic marker location in the complement of SEQ ID N^{o}2, is indicative of an increased risk that said patient consists of a good responder to interferon treatment with respect to standard responsiveness, and
(ii) a detection of a C/C homozygosity at said biallelic marker location in SEQ ID N^{o}2, or detection of a G/G homozygosity at said biallelic marker location in the complement of SEQ ID N^{o}2, is indicative of an increased risk that said patient consists of a non responder to interferon treatment with respect to standard responsiveness.

13. A method for treating a disease selected from the group consisting of Multiple Sclerosis disorder, hepatitis and cancer, comprising administering to a patient in need thereof, a recombinant human type I IFNβ interferon with reduced or no adverse side effects to the patient, and wherein said patient is one of:
(i) a patient, the genome of which exhibits a G/G homozygosity at said biallelic marker location in SEQ ID N^{o}1, or the genome of which exhibits a C/C homozygosity at said biallelic marker location in the complement of SEQ ID N^{o}1, and
(ii) a patient, the genome of which (1) exhibits a G/G homozygosity at said biallelic marker location in SEQ ID N^{o}1, or exhibits a C/C homozygosity at said biallelic marker location in the complement of SEQ ID N^{o}1, and which (2) further exhibits no C/C homozygosity at said biallelic marker location in SEQ ID N^{o}2, or further exhibits no G/G homozygosity at said biallelic marker location in the complement of SEQ ID N^{o}2.

14. The method according to claim 13, wherein the patient is affected with Multiple Sclerosis disorder.
